(19) United States
(12) Patent Application Publication
(10) Pub. No.: US 2010/0233397 A1
(43) Pub. Date: Sep. 16, 2010

(54) FRAGRANT EGAL AND METHOD FOR MAKING THE SAME

(76) Inventor: Mahmoud E. Al-Mutawa, Safat (KW)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/400,987

(22) Filed: Mar. 10, 2009

(51) Int. Cl. B32B 1/00; A61K 8/02; D03D 29/00

(52) U.S. Cl. 428/35.6; 512/4; 28/151

(57) ABSTRACT

A fragrant egal consists of an inner ring of aromatic material and a mass of cotton or sheep's wool wrapped around the aromatic ring. The sheep's wool and aromatic ring are then held together by tightly woven black goat's hair thread that is wrapped around the sheep's wool and long lasting fragrant material to form an endless rope like ring and worn as an adornment on the head of an individual and to hold a head scarf in place.

### FIELD OF THE INVENTION

This invention relates to a fragrant Egal and method for making the same and more particularly to a fragrant Egal or tightly woven endless rope like ring of wool and goat's hair worn as an outer adornment and to hold a scarf on the head of an individual, and to a method for making the fragrant Egal.

### BACKGROUND FOR THE INVENTION

An egal, sometimes spelled agal or igal is an accessory constructed of cord which is fastened around the Kofiyeh (Arabic scarf or head dress) to hold it in place. The egal is usually made of sheep's wool and tightly woven black goat's hair thread wrapped tightly around the sheep's wool to form a rope like ring. This ring or in practice a double ring is worn sitting on top of a man's head to hold a scarf in place and is not wrapped around the forehead like a hat.

The egal has been worn since ancient time as a symbol of dignity and prestige. The egal is regarded as complimentary to the dress, especially in the Arab Gulf States and in some Arabic countries. It is a symbol of cultural and social characteristic and has become on manifestation of a visual protocol and social events. Wearing the egal has become part of the personality, and one of the components and the basic of Arabic dress which is part of the history, identity and an extension of the pride in Arabic traditions.

A Kalensky, U.S. Pat. No. 2,709,261 discloses a turbine or headdress for women that are constructed with an annular ring shaped foundation for supporting scarfs and/or stringers on the top of the head. A more recent patent of Reynolds, U.S. Pat. No. 5,161,260 relates to an athletic headdress that includes an elastic headband integral with a front edge of a body portion and including a sweat pad.

In addition, a Nebeker, U.S. Pat. No. 5,566,395 discloses a disposal liner for a hat for absorbing moisture and oils and to eliminate odor. It also suggest that in addition to its use as a deodorizer for preventing and eliminating odor, the deodorizer may include an application of a fragrance.

Notwithstanding the above, it is presently believed that there is a need and a potential commercial market for a fragrant egal in accordance with the present invention. There should be a market for such egals because they impart a pleasant odor and eliminate the need for perfume, cologne, aftershave lotions and the like. Further, such fragrances are long lasting and the fragrance last for up to a year. Further, the fragrant egal does not come into contact with the skin or hair of an individual. Thus they do not cause irritation to the skin or change characteristics when coming into contact with body oils.

### BRIEF SUMMARY OF THE INVENTION

In essence the present invention contemplates a fragrant egal comprising a tightly woven endless rope like ring of sheep's wool or cotton (preferably sheep's wool) and woven black goat's hair thread wrapped tightly around the sheeps wool. A long lasting fragrant material that will not adversely affect the thickness of a traditional egal and will not hinder its flexibility is incorporated in the interior of the egal. A single or double ring is then worn on the head of an individual.

The invention also contemplates a method for making a fragrant egal that includes the steps of providing a mass of fragrant material such as sandlewood and a mass of cotton or sheep's wool and wrapping the fragrant material with the cotton or sheep's wool to form a rope like interior stuffing. A mass of goat's hair thread is wrapped around the interior stuffing to form a rope like structure having two ends. The rope like structure is sized and formed into an endless ring by stitching the two ends together. Any excess hair is burned off with direct fire for some models only (the cotton and sheep wool are dyed black in early stages before the final shape of ring like structure). A wood mold and wood mallet are provided and the dyed ring is placed on the mold and the ring hammered with the wood mallet to form a final shape. The fragrant egal is then washed and brushed (for those which need to burn, the hair to remove the burn smell) after which the fragrant Egal is ready for use.

The invention will now be described in connection with the accompanying drawings wherein like reference numbers have been used to indicate like elements.

### DESCRIPTION OF THE DRAWINGS

FIG. 1 is an endless ring of long lasting fragrant material for use in a first embodiment of the invention; if thickness of the Egal is slim

FIG. 2 illustrates a pair of endless rings of fragrant material as used in a preferred embodiment of the invention; if thickness of the Egal is thick

FIG. 3 is a partially spread apart portion of a fragrant egal in accordance with the first embodiment of the invention;

FIG. 4 is a schematic illustration showing the composition of a fragrant egal in accordance with the present invention;

FIG. 5 is a perspective view of a fragrant egal in accordance with the invention; and

FIG. 6 is a flow chart illustrating a method for making a fragrant egal in accordance with the present invention.

### DESCRIPTION OF THE PREFERRED EMBODIMENTS OF THE INVENTION

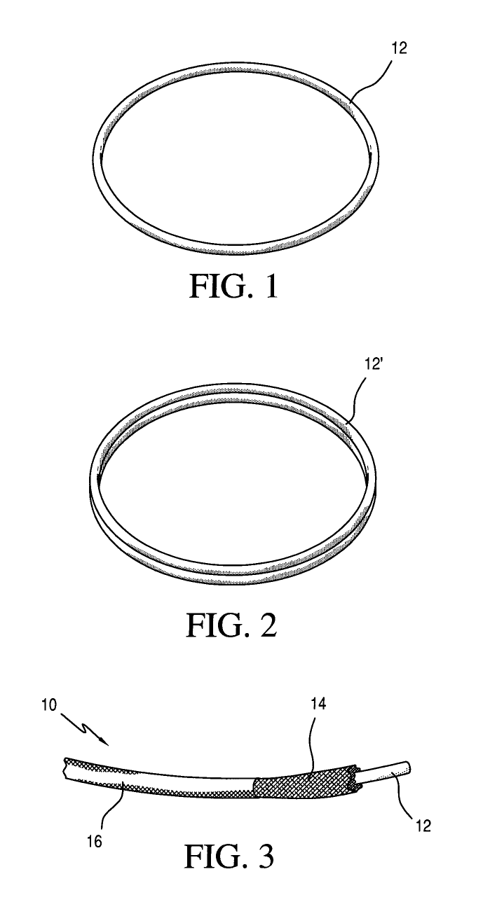

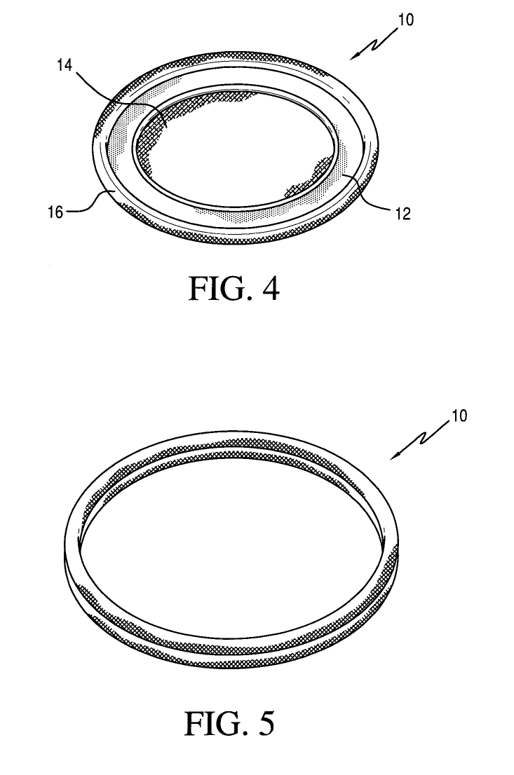

The construction of a fragrant egal 10 in accordance with the present invention is illustrated in FIGS. 1-5. As illustrated a first embodiment of the invention includes a base ring 12 of long lasting fragrant material, as for example sandalwood or other long lasting aromatic material that will provide an egal of the same thickness (diameter) with the flexibility of a traditional egal. The aromatic or fragrant material 12 may be cotton or raw material, which is impregnated with an Arabic mix of aromatic material, European aromatic material or both.

The fragrance is typically a complex blend of aromatic substances extracted from natural plants or animal products in addition to synthetic chemicals that also contain anti-oxidants to slow oxidation in the air as well as chemicals to avoid degradation as will be well understood by persons of ordinary skill in the art. The fragrant materials may also include a vegetable oil base and may include sandalwood, saffron, orange blossom, nutmeg or any other fragrance type as well as animal products that are typically used in perfumes and provide a fragrance that will last for a year or more.

In the construction of a fragrant egal 10 the fragrant material 12 is wrapped with a mass of cotton or wool thread 14 or other form and held in place by an external covering 16. The external covering 16 is thread made from goat or sheep hair or a synthetic thread as will be explained in a description of a method for making a fragrant egal, the goat's hair is dyed black.

Tools used in making an egal include “AL-TASKAAH”, a piece of wood (150 cm in length) with two cushions for making the lining and spinning the external layer of the egal; a pistol made from wood and used to tighten its circular shape, a spinner like a spindle to spin the threads, a template (mold) to size the egal, an iron brush to clean the outer layer, a hot iron, a needle and comb.

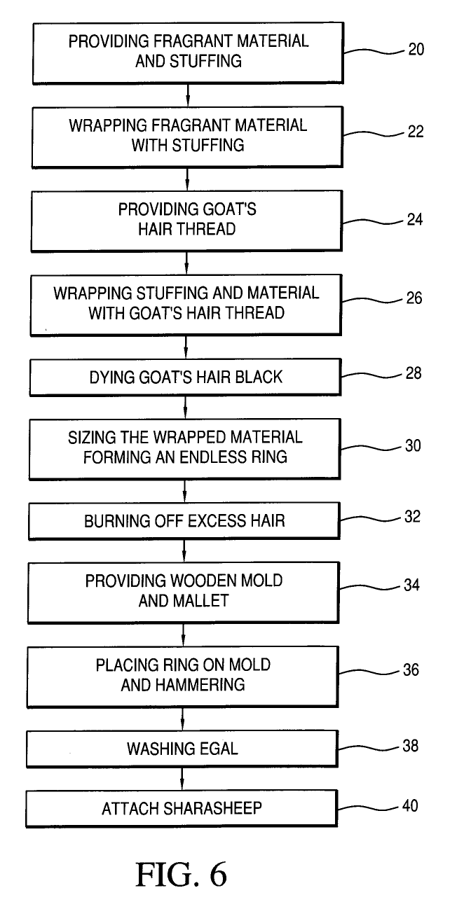

A method for making a fragrant egal in accordance with a preferred embodiment of the invention will now be described in connection with FIG. 6. As shown therein, the method for making a fragrant egal includes the step 20 of providing a mass of fragrant material as described above and a mass of cotton or wool stuffing material that may be in the form of spun thread. In step 20 is followed by a step 22 of wrapping and spinning the cotton or wool threads around the fragrant material to form a rope like structure with the fragrant material extending longitudinally through the center of the stuffing material. The amount of fragrant material and stuffing material are sufficient to provide a cross sectional thickness of a traditional egal, i.e. approximately 1 inch in diameter.

In steps 24 and 26 the mass of goat's hair thread is provided and wrapped tightly around the fragrant material and stuffing material. The goat's hair thread is dyed black in step 28 and can be done before wrapping it around the fragrant material and cotton or wool stuffing to form a tightly wrapped slightly flexible rope like structure. The rope like structure is sized leaving two ends in order to accommodate different sizes of an individual's head. Then the two ends of the rope like structure are joined together to form an endless ring in step 30. The two ends are sewn together with straight or curved needles with a method referred to as “Tadreeza” and wrapped in a similar matter as the ring with goat's hair thread.

A high temperature iron is then used in step 32 to burn off any excess hair for some types of Egals and a wooden form or mold and wooden mallet are provided in step 34. The endless ring is then placed on the wooden form or mold and hammered with the wooden mallet in step 36 to form a final determined shape. The hammered ring is then washed in step 38. At this point one has a final Egal that includes two rings bound together by the black goat's hair thread. However, in some cases a groups of threads called “Sharasheep” are added to the back of the Egal for decoration in an added step 40.

While the invention has been described in connection with its preferred embodiments it should be recognized that changes and modifications may be made therein without departing from the scope of the appended claims.

What is claimed is:

1. A fragrant egal comprising a tightly woven endless rope like ring of sheep's wool or cotton and woven black goat's hair thread wrapped tightly around said sheep's wool or cotton and a long lasting fragrant material that will not adversely effect the thickness of a traditional egal and will not hinder the flexibility thereof disposed in said interior thereof, and said egal adapted to be worn as an outer adornment and to hold a head scarf on the head of an individual.

2. A fragrant egal according to claim 1 in which said goat's hair is spun into a thread.

3. A fragrant egal according to claim 2 in which said fragrant material is a cotton plastic or solid stick like material that is formed into a ring.

4. A fragrant egal according to claim 3 in which said fragrant material is selected from the group consisting of sandal wood, saffron, essential oils, nutmeg or any other fragrance type and mixture thereof.

5. A fragrant egal consisting of a tightly woven rope like ring of sheep's wool and woven black goat's hair thread wrapped around said sheep's wool, and a long lasting fragrant material that will not adversely affect the thickness of a traditional egal and will not hinder the flexibility thereof, and said egal constructed and dimensional to be worn as an outer adornment and to hold a scarf onto the head of an individual.

6. A fragrant egal according to claim 5 that includes two rope like rings of sheep's wool with a tightly woven black goat's hair thread wrapped around said sheep's wool and a fragrant material disposed in the interior thereof.

7. A method for making a fragrant egal comprising the steps of:
providing a mass of fragrant material and a mass of cotton, wool thread or spunge thread mixed with polyester;
wrapping the fragrant material with a cotton or wool thread to form an interior stuffing;
providing a mass of goat's hair thread and wrapping the interior stuffing with the goat's hair thread to form a rope like structure having two ends;
sizing the wrapped stuffing and forming a ring from the rope like structure;
stitching the ends of the rope like structure to form an endless ring;
burning off any excess hair and dying the endless ring black;
providing a wooden mold and a wood mallet;
placing the endless ring on the mold and hammering the ring with the wooden mallet to form a final shape; and
washing the fragrant egal.

8. A method for making a fragrant egal according to claim 7 which includes the steps of providing a direct fire for some types of Egal to burn off excess goat's hair.

9. A method for making a fragrant egal according to claim 8 in which the stitching is done by a method called Tadreeza.

10. A method for making a fragrant egal according to claim 9 which includes the step of attaching a group of tassels called Sharasheep as further decoration.
